(19) United States
(12) Patent Application Publication
(10) Pub. No.: US 2024/0366733 A1
(43) Pub. Date: Nov. 7, 2024

(54) COLLAGEN BEVERAGES AND METHODS OF MAKING AND USING

(71) Applicant: Melaleuca, Inc., Idaho Falls, ID (US)

(72) Inventors: Jordan Joy, Idaho Falls, ID (US); Nasser A. Fredj, Idaho Falls, ID (US)

(21) Appl. No.: 18/654,712

(22) Filed: May 3, 2024

Related U.S. Application Data
(60) Provisional application No. 63/463,843, filed on May 3, 2023.

(51) Int. Cl. A61K 38/39; A23L 2/39; A23L 2/44; A23L 2/66; A61K 9/00; A61K 31/122; A61K 31/216; A61K 31/375; A61K 31/4188

(52) U.S. Cl. CPC A61K 38/39; A61K 8/35; A61K 8/65; A61K 9/0095; A61K 31/122; A61K 31/232; A61K 31/375; A61K 31/4188; A61Q 5/00; A61Q 19/00; A61K 2800/92

(57) ABSTRACT

This disclosure provides a beverage composition that includes collagen peptides that can be used to improve skin, joint and/or hair health.

### CROSS-REFERENCE TO RELATED APPLICATIONS

This application claims priority from U.S. Provisional Application Ser. No. 63/463,843, filed on May 3, 2023. The disclosure of the prior application is considered part of (and is incorporated by reference in) the disclosure of this application.

### TECHNICAL FIELD

This disclosure generally relates to collagen beverages and methods of making and using such collagen beverages.

### BACKGROUND

Skin is primarily composed of collagen proteins, which give skin its firmness and smoothness. However, the collagen content of skin declines with age and with UV exposure; age involves, in general, a progressive loss of function in the ability to make proteins, and UV exposure increases free radicals, which damage existing skin proteins such as collagen, reducing skin firmness and hydration.

Joints correspond to junctions between bones where articulation occurs. Cartilage, which is made from collagen proteins, is present in joints to protect the bones from wear. With advanced age, cartilage wears down and bones can begin to make contact, causing pain, reducing physical abilities, and compromising quality of life.

Consumers seek products that preserve their youth, including skin and joint health. Therefore, products that improve skin and joint health are highly desirable.

### SUMMARY

Provided herein is a beverage composition that includes particular collagen peptides. The beverage compositions described herein can be ingested to improve the health of skin, joints, and/or hair.

In one aspect, powdered beverage compositions are provided that include first collagen peptides, second collagen peptides, and axtaxanthin, wherein the first collagen peptides have an average molecular weight of about 1 kDa to about 4 kDa and the second collagen peptides have an average molecular weight of about 2 kDa to about 5 kDa. In another aspect, aqueous beverage compositions are provided that include first collagen peptides, second collagen peptides, axtaxanthin, and an aqueous carrier, wherein the first collagen peptides have an average molecular weight of about 1 kDa to about 4 kDa and the second collagen peptides have an average molecular weight of about 2 kDa to about 5 kDa.

In some embodiments, the first collagen peptides are present in the composition in an amount of from about 15 weight % to about 30 weight %. In some embodiments, the second collagen peptides are present in the composition in an amount of from about 35 weight % to about 55 weight %. In some embodiments, the astaxanthin is present in the composition in an amount of from about 0.5 weight % to about 2.5 weight %.

In some embodiments, the composition further includes one or more vitamins. In some embodiments, the one or more vitamins include vitamin A palmitate. In some embodiments, the vitamin A palmitate is present in the composition in an amount of from about 0.01 weight % to about 0.1 weight %. In some embodiments, the one or more vitamins includes biotin. In some embodiments, the one or more vitamins includes ascorbic acid. In some embodiments, the ascorbic acid is present in the composition in an amount of from about 0.1 weight % to about 1 weight %. In some embodiments, the aqueous carrier is water (e.g., flavored water).

In another aspect, methods of improving skin, joint, and/or hair health are provided. Such methods typically include solubilizing the powdered beverage composition described herein into an aqueous carrier; and ingesting an effective amount of the solubilized beverage composition, thereby improving skin, joint, and/or hair health.

In yet another aspect, methods of improving skin, joint, and/or hair health are provided. Such methods typically include ingesting an effective amount of the aqueous beverage composition described herein, thereby improving skin, joint, and/or hair health.

In still another aspect, methods of making the beverage mix described herein are provided. Such methods typically include combining the first collagen peptides, the second collagen peptides, and the astaxanthin. In some embodiments, the methods further include adding an aqueous carrier.

In another aspect, powdered beverage composition are provided that include collagen peptides, astaxanthin, biotin, and at least one antioxidant, wherein the collagen peptides have an average molecular weight of about 1 kDa to about 5 kDa.

In still another aspect, powdered beverage compositions are provided that include first collagen peptides, second collagen peptides, and at least one antioxidant, wherein the first collagen peptides have an average molecular weight of about 1 kDa to about 4 kDa and the second collagen peptides have an average molecular weight of about 2 kDa to about 5 kDa. In some embodiments, the at least one antioxidant is selected from vitamin A and vitamin C.

In yet another aspect, aqueous beverage compositions are provided that include first collagen peptides, second collagen peptides, at least one antioxidant, and an aqueous carrier, wherein the first collagen peptides have an average molecular weight of about 1 kDa to about 4 kDa and the second collagen peptides have an average molecular weight of about 2 kDa to about 5 kDa. In some embodiments, the at least one antioxidant is selected from vitamin A and vitamin C.

In one aspect, powdered beverage compositions are provided that include first collagen peptides, second collagen peptides, and biotin, wherein the first collagen peptides have an average molecular weight of about 1 kDa to about 4 kDa and the second collagen peptides have an average molecular weight of about 2 kDa to about 5 kDa.

In yet another aspect, aqueous beverage compositions are provided that include first collagen peptides, second collagen peptides, biotin, and an aqueous carrier, wherein the first collagen peptides have an average molecular weight of about 1 kDa to about 4 kDa and the second collagen peptides have an average molecular weight of about 2 kDa to about 5 kDa.

In yet another aspect, powdered beverage mix composition contained in a bulk package are provided, wherein the bulk package containing a measuring scoop and the powdered beverage mix composition, the measuring scoop being sized to scoop an amount of the powdered beverage mix composition suitable for blending with a liquid to form a beverage. Typically, amount of the powdered beverage mix composition is 10 to 20 g and comprises: a) between 15 weight % and 30 weight % of first collagen peptides, wherein the first collagen peptides have an average molecular weight of about 1 kDa to about 4 kDa; b) between 35 weight % and 55 weight % of second collagen peptides, wherein the second collagen peptides have an average molecular weight of about 2 kDa to about 5 kDa; and c) between 0.5 weight % and 2.5 weight % astaxanthin.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DETAILED DESCRIPTION

This disclosure describes a collagen-based beverage formula that utilizes collagen peptides that are biologically active for increasing endogenous collagen production site-specifically within the skin and joints. In addition, the composition also includes natural astaxanthin to provide anti-oxidant benefits.

### Collagen

The first ingredient is collagen peptides having an average molecular weight of about 1 kDa to about 4 kDa, which have been shown to increase collagen mRNA and fibroblast cell stimulation two-times greater than that of generic collagen peptides. Generally, between about 1 g and about 10 g (e.g., about 2 g to about 7.5 g, about 2.5 g to about 5 g, about 3 g to about 4 g, about 1 g to about 5 g, about 1 g, about 3 g, about 5 g, about 10 g) of the first collagen peptides is used in a composition as described herein. The first collagen peptides can take any number of forms including, for example, a hydrolyzed powder. A skilled artisan would be able to generate collagen peptides having an average molecular weight in the range of about 1 kDa to about 4 kDa using known methods.

Similarly, the second ingredient also is collagen peptides, but having an average molecular weight of about 2 kDa to about 5 kDa, which have been shown to stimulate collagen production in the chondrocytes at a rate of twice that of other collagen peptides. Generally, between about 2.5 g and about 20 g (e.g., about 3 g to about 15 g, about 5 g to about 10 g, about 6 g to about 8 g, about 3 g, about 5 g, about 10 g, about 15 g, about 20 g) of the second collagen peptides is used in a composition as described herein. As above, the second collagen peptides can take any number of forms including, for example, a hydrolyzed powder. A skilled artisan would be able to generate collagen peptides having an average molecular weight of about 2 kDa to about 5 kDa using known methods.

Without being bound by theory, it is believed that both the first collagen peptides and the second collagen peptides bind to integrins on cell membranes and activate collagen production within the target cells, irrespective of the type of collagen (I, II, III, V, or otherwise) from which it is derived. Existing collagen products primarily use collagen peptides made from a non-specific enzymatic hydrolysis, which have not been found to independently improve skin health and anti-aging. On the other hand, the first and second collagen peptides have been shown to reduce wrinkles, cellulite, and joint discomfort in human trials while increasing cartilage production in the joints. The first collagen peptide product (e.g., about 2 kDa collagen peptides) has been shown to improve skin health and reduce cellulite at 2.5 g daily. 10 g of generic collagen does have some studies to support it for improving joint comfort, however, the same can be achieved with just 5 g of the second collagen peptides (e.g., about 3 kDa collagen peptides), which also has been shown to increase cartilage regeneration at 10 g.

In some instances, standard collagen peptides having an average molecular weight of about 1 kDa to about 5 kDa can be used in a beverage composition as described herein. Exemplary standard collagen peptides include, without limitation, Peptiplus® or Peptiplus XB aggl® (Gelita), but a skilled artisan would be able to generate standard collagen peptides having an average molecular weight of about 1 kDa to about 5 kDa using known methods. About 1 g to about 10 g (e.g., about 2.5 g to about 7.5 g, about 3 g to about 6 g, about 5 g, about 2.5 g) of standard collagen peptides can be used in a beverage composition as described herein.

### Astaxanthin

Astaxanthin is an antioxidant with a unique size, shape, and physical properties that allow it to position itself across the cell membrane to protect the cell from free radical damage inside and out. Astaxanthin helps protect cells from photoaging and UV-induced oxidative stress. The optional addition of astaxanthin in the compositions described herein reduces matrix metalloproteinase content, which reduces the breakdown of skin collagen and elastin, allowing skin to maintain better firmness and hydration through improved barrier function and reduced water loss. Astaxanthin also has been observed to decrease sensitivity to UV exposure, reducing redness (versus placebo) after exposure to a fixed duration and intensity of light. Generally, between about 2 mg and about 12 mg (e.g., about 3 mg to about 8 mg, about 4 mg to about 9 mg, about 6 mg to about 8 mg, about 5 mg to about 10 mg, about 6 mg to about 12 mg) of astaxanthin can be used in a composition as described herein, and the astaxanthin can be natural, synthetic or combinations thereof, provided the functionality and efficacy remains unchanged. Few products exist on the market that combine a specialized collagen with astaxanthin, and this is the only one using efficacious doses of both. Together, the collagen peptides described herein and the optional astaxanthin work to increase collagen production and reduce collagen breakdown, respectively.

The efficacy of the collagen peptides and the astaxanthin can be enhanced by additional ingredients or delivery technologies.

### Vitamin A Palmitate (VAP)

Vitamin A palmitate (VAP) is the ester of retinol and palmitic acid, and can be converted into retinol, and ultimately into retinoic acid. Vitamin A is a fat-soluble polyunsaturated hydrocarbon vitamin that includes retinoids and carotenoids. Vitamin A obtained from plant source is a carotenoid that the human body can transform into a retinol, while the vitamin A from animal sources is already in a form of retinol that is easily absorbed by the body. Palmitic acid is a 16-carbon saturated fatty acid, and is typically considered to be the most common saturated fatty acid found in animals, plants and microorganisms. Vitamin A and VAP can be used for their antioxidant activity.

The degradation of VAP in aqueous solution is rapid, and oxygen accelerates photo-catalyzed degradation of retinoids under light or chemically generated free radicals. In addition, the solubility of retinoids in aqueous solvents is poor because of their low polarity. Therefore, there is a need to deliver VAP in a form that protects it from chemical degradation during storage. Dispersibility and stability can be improved by encapsulating VAP oil in powder microcapsules by using suitable barrier component. Different techniques can be used to encapsulate VAP including, for example, spray drying of a formulated emulsion or suspension is a widely used technique for preparation of microcapsules.

For spray drying processes, carbohydrates, gums, semisynthetic cellulose derivatives and synthetic polymers are commonly used carriers. Maltodextrin is a hydrolyzed starch having multiple functions including film formation properties, binding ability, and reduction of oxygen permeability of the barrier component. Maltodextrin, however, has a low glass transition temperature and low emulsifying capacity to agglomeration or caking of micro particulate powders. Therefore, to increase the efficiency of microencapsulation, combinations of barrier materials are often used.

Vitamin A or VAP can be used in a composition as described herein in an amount between about 300 IU to about 6000 IU (e.g., about 500 IU to about 5000 IU, about 1000 IU to about 3000 IU, about 1250 IU to about 1750 IU, about 1500 IU).

### Biotin

Biotin is one of the B vitamins (i.e., vitamin B7). Biotin is involved in a wide range of metabolic processes in humans and other organisms, primarily related to the utilization of fats, carbohydrates, and amino acids. Biotin is water soluble and is classified as a heterocyclic compound, with a sulfur-containing ring. Biotin is a co-enzyme for carboxylase enzymes, which are involved in the synthesis of fatty acids, the breakdown of amino acids and fatty acids, and gluconeogenesis. In addition, biotinylation of histone proteins in chromatin plays a role in nucleic acid stability and gene expression.

Biotin can be used in a composition as described herein in an amount between about 0.01 mg and about 0.06 mg (e.g., about 0.02 mg to about 0.05 mg, about 0.03 mg to about 0.04 mg, about 0.03 mg).

### Ascorbic Acid

Ascorbic acid is a chemical compound (C6H806) commonly known as vitamin C that is found in nature and can be used as an antioxidant additive in food and drink. Slowing the oxidation preserves color and freshness. The low pH of ascorbic acid can help prevent microbial growth, thereby preventing spoilage and preserving freshness. For these reasons, ascorbic acid is a popular natural ingredient preservative.

Ascorbic acid can be used in a variety of forms, including salts and esters. In these forms, it will appear on ingredient lists under different names, such as sodium ascorbate, calcium ascorbate, potassium ascorbate, ascorbyl palmitate, or ascorbyl stearate. Ascorbic acid can be used in a composition as described herein in an amount between about 9 mg and about 450 mg (e.g., about 18 mg to about 360 mg, about 25 mg to about 180 mg, about 40 mg to about 100 mg, about 45 mg, about 55 mg, about 90 mg).

### Additional Ingredients

Any number of additional ingredients can be used in the compositions described herein for purposes of, without limitation, emulsifiers, solubilizers, flavorings, colorings, stabilizers, sweeteners, and/or minerals. Representative ingredients that can be included in a composition as described herein include, for example, salts (e.g., sodium chloride) and/or silicon dioxide.

Salts are used in beverages to enhance sweetness and balance bitterness. For example, salts can enhance citrus flavors and also can add depth and texture in carbonated beverages.

Silicon dioxide is used in the food and beverage industries, both as additives and as flavoring intermediates. Silicon dioxide can help in the flow and mixing of substances during production, and can absorb moisture in raw materials and in the air to protect important materials (e.g., medicines). Silicon dioxide is used as an anti-caking agent in powdered beverages such as milk powder, fruit juice powder, powdered drinks, and seasoning powder. An important property of the anti-caking agent is that the silicon dioxide absorbs water or binds very well to the water. Silicon dioxide may absorb up to 2 times the weight of body weight. It helps to absorb water from the atmosphere that surrounds the food in the package and helps to absorb water from the surface of the powdered food as well.

Representative ingredients that can be included in a composition as described herein also include, for example, calcium (e.g., as calcium carbonate, dicalcium phosphate, calcium citrate, calcium gluconate, calcium amino acid polysaccharide complex); iron (e.g., as an iron amino acid polysaccharide complex), phosphorus (e.g., as dicalcium phosphate), iodine (e.g., as iodine amino acid polysaccharide complex), magnesium (e.g., as magnesium oxide, magnesium lactate, magnesium citrate, magnesium amino acid polysaccharide complex), zinc (e.g., as a zinc amino acid polysaccharide complex), selenium (e.g., as a selenium amino acid polysaccharide complex), copper (e.g., as a copper amino acid polysaccharide complex), manganese (e.g., as a manganese amino acid polysaccharide complex), chromium (e.g., as a chromium amino acid polysaccharide complex), molybdenum (e.g., as a molybdenum amino acid polysaccharide complex), boron (e.g., as a boron amino acid polysaccharide complex), vitamin D (e.g., as cholecalciferol), vitamin E (e.g., as d-alpha tocopheryl succinate), vitamin K (e.g., as phytonadione), thiamin (e.g., as thiamin HCl), riboflavin, niacin (e.g., as niacinamide), vitamin B6 (e.g., as pyridoxine HCl), folate (e.g., as folic acid or methyl folate), vitamin B12 (e.g., as cyanocobalamin or methylcobalamin), and combinations thereof.

Any additional ingredients can be used in a composition as described herein in an amount ranging between trace amounts up to about 5 g (e.g., about 0.01 mg to about 1 g, about 1 mg to about 100 mg).

### Methods of Making a Collagen Drink Composition

The collagen-containing compositions described herein can be formulated for oral administration. In one embodiment, collagen-containing compositions described herein can be formulated as a powdered beverage mix that can be mixed with a pharmaceutically acceptable aqueous carrier by the user, or collagen-containing compositions described herein can be formulated as an already mixed beverage.

Suitable aqueous carriers include, for example, water, alcohols, saline, and buffered solutions. Suitable aqueous carriers also can include physiologically acceptable aqueous vehicles (e.g., physiological saline) or other carriers appropriate for oral administration.

In some cases, liquid preparations can contain pharmaceutically acceptable additives such as suspending agents, emulsifying agents, non-aqueous vehicles, preservatives, buffer salts, flavoring agents, coloring agents, sweetening agents, suitable excipients, flavorings, colorants, and other ingredients as appropriate. For oral administration, one or more pharmaceutically acceptable excipients or carriers can be used such as binding agents, fillers, lubricants, disintegrants, or wetting agents.

In some cases, the collagen peptides described herein can be mixed with an aqueous carrier (e.g., water) at room temperature, while, in some cases, the collagen peptides described herein can be mixed with an aqueous carrier (e.g., water) in the presence of heat (about 22° C. to about 82° C., about 22° C., about 27° C., about 32° C., about 37° C., about 42° C., about 47° C., about 47° C., about 52° C., about 57° C., about 62° C., about 67° C., about 72° C., about 77° C., about 82° C., about 22° C. to about 27° C., about 27° C. to about 32° C., about 32° C. to about 37° C., about 37° C. to about 42° C., about 42° C. to about 47° C., about 47° C. to about 52° C., about 52° C. to about 57° C., or about 57° C. to about 62° C., about 62° C. to about 67° C., about 67° C. to about 72° C., about 72° C. to about 77° C., or about 77° C. to about 82° C.). If heating is necessary, the mixture of collagen peptides and water can be heated for about 5 minutes to about 30 minutes (e.g., about 5 minutes to about 10 minutes, about 10 minutes to about 15 minutes, about 15 minutes to about 20 minutes, about 20 minutes to about 25 minutes, or about 25 minutes to about 30 minutes).

“Dose” or “total dose” refers to the amount of ingredient(s) administered over a designated period of time. For example, a “daily dose” would be understood to be the amount of ingredient(s) administered over a 24 hour period of time in a suitable volume. A total daily dose can be prepared for administration in the form of one or more dosage units (e.g., two, three, or four beverages). In some cases, the one or more dosage units can be administered in one or more dosages over the course of 24 hours (e.g., one dose, two doses, or three doses), where the total amount of ingredient(s) in the one or more dosages does not exceed the total daily dose.

In some cases, a serving can be at least 8 g of the beverage mix in powder form. In some cases, a serving of the beverage mix composition can be about 8 g to about 20 g, about 10 g to about 18 g, about 12 g to about 16 g, about 12 g to about 14 g, or about 10 g to about 14 g, in powder form. In some cases, a serving of the beverage mix composition can be about 10 g to about 14 g in powder form. In some cases, a serving of the beverage mix composition can be about 12 g in powder form.

In some cases, one serving of the beverage mix composition in powder form can be dissolved into at least 8 fluid ounces of liquid (e.g., water or any other suitable beverage). In some cases, a serving of the beverage mix composition in powder form can be dissolved in about 8 fluid ounces to about 24 fluid ounces of liquid, about 10 fluid ounces to about 20 fluid ounces of liquid, about 12 fluid ounces to about 20 fluid ounces of liquid, about 14 fluid ounces to about 20 fluid ounces of liquid, about 14 fluid ounces to about 18 fluid ounces of liquid, or about 15 fluid ounces to about 17 fluid ounces of liquid. In some cases, a serving of the beverage mix composition in powder form can be dissolved in about 8 fluid ounces of liquid to about 20 fluid ounces of liquid. In some cases, a serving of the beverage mix composition in powder form can be dissolved in about 16 fluid ounces of liquid.

In some cases, a container or package containing a dry powder beverage mix composition provided herein can include a measuring scoop or spoon sized to measure out a single serving of a dry powder beverage mix composition provided herein. For example, a package (e.g., a jar, pouch, or any other suitable container) can contain multiple servings of a dry powder beverage mix composition provided herein, and can be provided to a subject in combination with a measuring spoon, a scoop, or any other device suitable for measuring out a single serving of a dry powder beverage mix composition provided herein. In some cases, a single serving of a dry powder beverage mix composition provided herein can be packaged in a single serving packet, pouch, or any other appropriate container from which the dry powder beverage mix composition can be dispensed into a fluid (e.g., water or another beverage) before consumption. In some cases, a plurality of such single serving containers can be packaged together (e.g., in a box or a larger pouch).

### Methods of Improving Skin, Joint and or Hair Health

In some cases, the methods and compositions provided herein can be used to improve skin, joint and/or hair health in a mammal (e.g., a human). The methods can include administering to a mammal an effective amount of a composition provided herein, where the composition includes the collagen peptides described herein. An effective amount of a composition can be, for example, a beneficial daily dose as set forth herein.

Skin health can be evaluated using any number of criteria. For example, a lack of blemishes (e.g., pimples, acne, blackheads), elasticity, firmness, smooth texture and/or even color can be used to evaluate the healthiness of skin.

Hair health can be evaluated using any number of criteria. For example, the health of hair can be evaluated based on the rate of growth, fullness, luster, sheen, the amount of breakage, and/or the amount of moisture.

In some embodiments, skin, joint and/or hair health can determined before ingesting the compositions described herein and then again after the composition has been ingested for a period of time (e.g., 2 weeks, one month, 6 months).

In accordance with the present invention, there may be employed biology, microbiology, and biochemical techniques within the skill of the art. Such techniques are explained fully in the literature. The invention will be further described in the following examples, which do not limit the scope of the methods and compositions of matter described in the claims.

### EXAMPLES

It would be understood that certain ingredients in the recipes shown below such as, without limitation, sodium chloride, are optional (i.e., may be removed from any of the recipes shown below).

Any of the suppliers and/or branded materials described herein may change; those shown in any of the Tables below are representative only.

### Example 1-First Exemplary Collagen Drink Mix

Table 1 shows a representative recipe for a collagen beverage mix as described herein.

TABLE 1
First Exemplary Collagen Drink Mix

Ingredient
Bovine Collagen Peptides
Bioactive Collagen Peptides (1-4 kDa)
Ascorbic Acid
Sodium Chloride
Retinol Palm Vit A Spry Dry
Biotin
2.5% Astaxanthin
90% Hydrolyzed Keratin Peptide

### Example 2-Second and Third Exemplary Collagen Drink Mixes

Tables 2 and 3 show additional representative recipes for collagen beverage mixes as described herein.

TABLE 2
Second Exemplary Collagen Drink Mix

| | Label Claim | Label |
|---|---|---|
| Ingredient | (UM/Serving) | UOM |
| Bioactive Collagen Peptides (2-5 kDa) | 10000 | mg |
| Bioactive Collagen Peptides (1-4 kDa) | 2500 | mg |
| Ascorbic acid | 45 | mg |
| Sodium chloride | 100 | mg |
| Retinol Palm Vitamin A Spry Dry | 1500 | IU |
| Biotin | 0.03 | mg |

TABLE 3
Third Exemplary Collagen Drink Mix

| | Label Claim | Label |
|---|---|---|
| Ingredient | (UM/Serving) | UOM |
| Bioactive Collagen Peptides (2-5 kDa) | 10000 | mg |
| Bioactive Collagen Peptides (1-4 kDa) | 2500 | mg |
| Ascorbic acid | 45 | mg |
| Sodium chloride | 100 | mg |
| Retinol Palm Vitamin A Spry Dry | 1500 | IU |
| Biotin | 0.03 | mg |
| 2.5% Astaxanthin | 4 | mg |

### Example 3 Fourth Exemplary Collagen Drink Mix

Table 4 shows a representative detailed recipe for a collagen beverage mix as described herein.

TABLE 4
Detailed Fourth Exemplary Collagen Drink Mix

| | Label Claim | Label | % | % | Input | Wt |
|---|---|---|---|---|---|---|
| Ingredient | (UM/Serving) | UOM | Activity | Overage | (mg) | (%) |
| Bioactive Collagen | 5000 | mg | 91 | 2 | 5618.13 | 35-55 |
| Peptides (1-4 kDa) | | | | | | |
| Bioactive Collagen | 2500 | mg | 91 | 2 | 2809.07 | 15-30 |
| Peptides (2-5 kDa) | | | | | | |
| Bovine Collagen | 2780 | mg | 91 | 2 | 3116.04 | 20-30 |
| (1-5 kDa) | | | | | | |
| Ascorbic acid | 45 | mg | 98 | 20 | 55.10 | 0.1-1 |
| Sodium chloride | 55 | mg | 44 | 0 | 125.00 | 0.5-2 |
| Retinol Palm | 1500 | IU | 25000 | 20 | 7.20 | 0.01-0.1 |
| Vitamin A Spry Dry | | | | | | |
| 2.5% Astaxanthin | 4 | mg | 2.5 | 20 | 192.00 | 0.5-2.5 |
| (water soluble) | | | | | | |
| Silicon dioxide | 77.5 | | | | 77.50 | 0.1-1 |

### Example 4 Results of Ingesting Collagen Drink Mix

Dry mix is relatively tasteless when mixed with a flavored drink product. Consumers ingesting a collagen beverage as described herein regularly (e.g., daily for at least a few weeks (e.g., a month)) report improved mobility with the same or less pain than prior to starting the collagen beverage regimen. Consumers ingesting a collagen beverage regularly as described herein also report feeling less joint pain and fuller, thicker hair than prior to starting the collagen beverage regimen. In some instances, consumers report a reduction in hair loss.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

What is claimed is:

1. A powdered beverage composition, comprising first collagen peptides, second collagen peptides, and axtaxanthin, wherein the first collagen peptides have an average molecular weight of about 1 kDa to about 4 kDa and the second collagen peptides have an average molecular weight of about 2 kDa to about 5 kDa.

2. The powdered beverage composition of claim 1, wherein the first collagen peptides are present in the composition in an amount of from about 15 weight % to about 30 weight %.

3. The powdered beverage composition of claim 1, wherein the second collagen peptides are present in the composition in an amount of from about 35 weight % to about 55 weight %.

4. The powdered beverage composition of claim 1, wherein the astaxanthin is present in the composition in an amount of from about 0.5 weight % to about 2.5 weight %.

5. The powdered beverage composition of any of the preceding claims, further comprising one or more vitamins.

6. The powdered beverage composition of claim 5, wherein the one or more vitamins comprises vitamin A palmitate.

7. The powdered beverage composition of claim 6, wherein the vitamin A palmitate is present in the composition in an amount of from about 0.01 weight % to about 0.1 weight %.

8. The powdered beverage composition of claim 5, wherein the one or more vitamins comprises biotin.

9. The powdered beverage composition of claim 5, wherein the one or more vitamins comprises ascorbic acid.

10. The powdered beverage composition of claim 9, wherein the ascorbic acid is present in the composition in an amount of from about 0.1 weight % to about 1 weight %.

11. The powdered beverage composition of any of the preceding claims, wherein the aqueous carrier is water.

12. The powdered beverage composition of any of the preceding claims, wherein the aqueous carrier is a flavored water.

13. A powdered beverage composition, comprising collagen peptides, astaxanthin, biotin, and at least one antioxidant, wherein the collagen peptides have an average molecular weight of about 1 kDa to about 5 kDa.

14. A powdered beverage composition, comprising first collagen peptides, second collagen peptides, and at least one antioxidant, wherein the first collagen peptides have an average molecular weight of about 1 kDa to about 4 kDa and the second collagen peptides have an average molecular weight of about 2 kDa to about 5 kDa.

15. The powdered beverage composition of claim 14, wherein the at least one antioxidant is selected from vitamin A and vitamin C.

16. A powdered beverage composition, comprising first collagen peptides, second collagen peptides, and biotin, wherein the first collagen peptides have an average molecular weight of about 1 kDa to about 4 kDa and the second collagen peptides have an average molecular weight of about 2 kDa to about 5 kDa.

17. A powdered beverage mix composition contained in a bulk package, the bulk package containing a measuring scoop and the powdered beverage mix composition, the measuring scoop being sized to scoop an amount of the powdered beverage mix composition suitable for blending with a liquid to form a beverage, wherein the amount of the powdered beverage mix composition is 10 to 20 g and comprises:
a) between 15 weight % and 30 weight % of first collagen peptides, wherein the first collagen peptides have an average molecular weight of about 1 kDa to about 4 kDa;
b) between 35 weight % and 55 weight % of second collagen peptides, wherein the second collagen peptides have an average molecular weight of about 2 kDa to about 5 kDa; and
c) between 0.5 weight % and 2.5 weight % astaxanthin.

18. A method of improving skin, joint, and/or hair health, comprising:
solubilizing the powdered beverage composition of claim 1 into an aqueous carrier; and
ingesting an effective amount of the solubilized beverage composition, thereby improving skin, joint, and/or hair health.

19. A method of making the beverage mix of claim 1, comprising:
combining the first collagen peptides, the second collagen peptides, and the astaxanthin.

20. The method of claim 19, further comprising adding an aqueous carrier.
